(19) United States
(12) Patent Application Publication
(10) Pub. No.: US 2012/0288906 A1
(43) Pub. Date: Nov. 15, 2012

(54) Process For Obtaining Fatty Acid Lower Alkyl Esters From Unrefined Fats And Oils

(75) Inventors: Horst Josten, Dusseldorf (DE); Ewelina Sobierska, Dusseldorf (DE); Adrian Chan, Dusseldorf (DE); Truc Tran Anh, Dusseldorf (DE)

(73) Assignee: Cognis IP Management GmbH, Dusseldorf (DE)

(21) Appl. No.: 13/470,282

(22) Filed: May 12, 2012

Related U.S. Application Data
(60) Provisional application No. 61/485,773, filed on May 13, 2011.

(51) Int. Cl. C07C 67/48; C12M 1/00; B01J 19/24; C12P 7/64

(52) U.S. Cl. 435/134; 554/170; 435/289.1; 422/187

(57) ABSTRACT

Described is a process for obtaining fatty acid C₁-C₄ alkyl esters from unrefined fats and oils, wherein:

(a) unrefined fats or oils having an acid value of from 1 to 20 are treated with hot steam in a counter-current column to provide a first fraction of free fatty acids and low boiling impurities and a second fraction of de-acidified and de-watered fats or oils;
(b) said first fraction is subjected to esterification with lower C₁-C₄ alcohols to provide a third fraction of fatty acid C₁-C₄ alkyl esters;
(c) said second and said third fraction are combined and subjected to a low pressure transesterification to provide an intermediate fraction of fatty acid C₁-C₄ alkyl esters, C₁-C₄ alcohols and glycerol; and
(d) said intermediate is subjected to a separation process to remove C₁-C₄ alcohols and the glycerol to provide a second fraction of C₁-C₄ alkyl esters.

### CROSS-REFERENCE TO RELATED APPLICATIONS

This application claims priority under 35 U.S.C. §119(e) to U.S. Provisional Application No. 61/485,773, filed May 13, 2011, the disclosure of which is incorporated herein by reference in its entirety.

### FIELD OF THE INVENTION

The present invention is related to the area of oleochemicals and refers to an improved process for obtaining fatty acid lower alkyl esters from unrefined fats and oils having acid values of at least 1.

### BACKGROUND

State of the art technologies for the manufacturing of methyl esters from vegetable oils and fats are low or high pressure transesterification with methanol and suitable catalysts. While for the low pressure transesterification with e.g. sodium methylate as catalyst refined oils with low free fatty acid content are required as feed materials, high pressure transesterification with e.g. Zn catalysts is able to convert unrefined oils and other low quality fats with high free fatty acid content.

A major advantage of the low pressure transesterification technology is related to the lower processing costs, in particular lower energy consumption, lower alcohol excess, lower maintenance costs and lower investment costs, if compared to high pressure transesterification. On the other hand the high pressure transesterification has the advantage, that cheaper raw materials and waste fat streams can be converted.

US patent application US 2009 0294358 A1 (Bayer) also discloses a process for transforming unrefined oils into lower fatty acid alkyl esters combining a pre-esterification step with a low pressure transesterification. However, the results from this process are not satisfying for the following reasons:
(i) Since the pre-esterification step is applied to the complete starting material huge esterification equipments are necessary;
(ii) Since crude vegetable oils contain solids, phospholipids and other unsaponifiable material the lifetime of the catalysts in the pre-esterification step is rather short;
(iii) The de-acidified crude oil still contains 0.2 to 0.3% water, therefore the consumption of catalyst (sodium hydroxide, potassium methylate or sodium methylate) in the subsequent low pressure trans-esterification is pretty high, since 0.1% water consumes as much catalyst as 1% fatty acids. At the same time the yield of methyl esters yield loss via soap formation (reaction of catalyst/water with methyl ester and glycerides) is significant.

Therefore, there is a need for improved processes for producing fatty acid lower alkyl esters.

### BRIEF DESCRIPTION OF THE DRAWINGS

FIG. 1 shows a flow sheet of a process comprising de-acidification, transesterification and separation steps in accordance with one or more embodiments of the invention.

### DETAILED DESCRIPTION

Embodiments of the present invention provide a process combining the advantages of both state of the art processes while avoiding their disadvantages, in particular providing a process for obtaining fatty acid lower alkyl esters from cheap raw materials, as for example natural triglycerides showing acid values of at least 1, and involving a low pressure instead of an high pressure transesterification step. In addition, in some embodiments the desired products are obtained in a continuous process.

One or more embodiments of the present invention relate to a process for obtaining fatty acid C₁-C₄ alkyl esters, preferably methyl esters, from unrefined fats and oils, wherein:
(a) unrefined fats or oils having an acid value of from 1 to 20, preferably 5 to 15 are treated with hot steam in a counter-current column to provide a first fraction of free fatty acids and low boiling impurities at the top and a second fraction of de-acidified and de-watered fats or oils at the bottom of the column;
(b) said first fraction is subjected to esterification with one or more lower C₁-C₄ alcohols in order to provide a third fraction of fatty acid C₁-C₄ alkyl esters;
(c) said second and said third fraction are combined and subjected to a low pressure transesterification in order to provide an intermediate fraction of fatty acid C₁-C₄ alkyl esters, C₁-C₄ alcohols and glycerol; and
(d) said intermediate is subjected to a separation process in order to remove C₁-C₄ alcohols and the glycerol in order to provide a second fraction of C₁-C₄ alkyl esters.

While the process can involve all kinds of lower C₁-C₄ alcohols, like ethanol and the isomeric propyl and butyl alcohols, in some embodiments the C₁-C₄ alcohol is methanol such that methyl esters are produced.

Surprisingly it has been observed that a process involving steps (a) to (d) provides many advantages. In particular, one major advantage of the process according to the invention with respect to low pressure transesterification processes applied to refined oils as known from the state of the art is the higher economy, which is due to the use of the cheaper crude oil. With respect to high pressure transesterification the advantages are:
lower energy consumption due to lower temperatures and lower alcohol excess in the transesterification step and due to simplified alcohol recovery (in particular flash evaporation instead of fractionation column, since water content in alcohol is less than 0.2%);
higher yields of alkyl esters due to lower soap formation and lower product degradation due to lower reaction temperature;
simplified procedure for alcohol recovery from the reaction mixture (flash distillation instead of column);
overall lower investment costs.

Step a: De-Acidification of Crude Oils

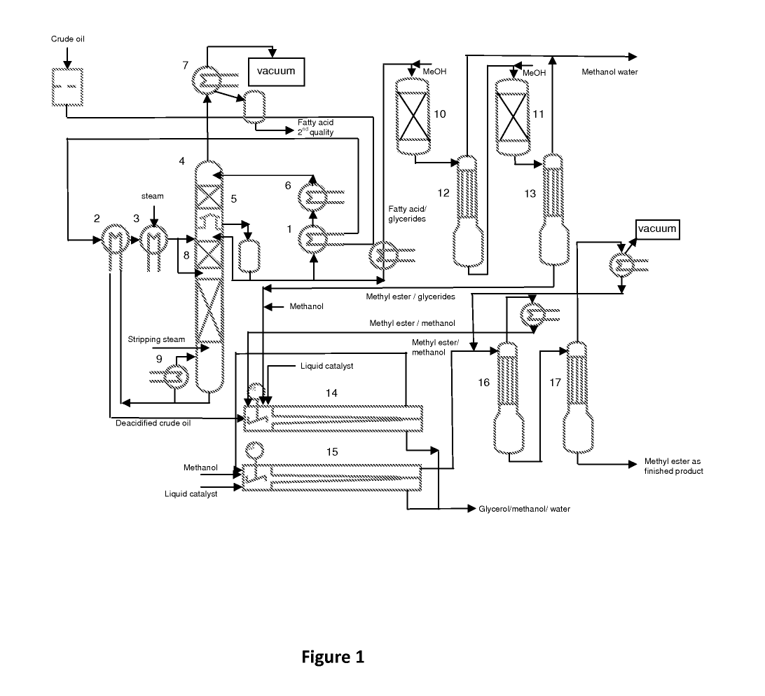

The process according to the present invention can be applied to all kinds of vegetable oils, in particularly those oils showing an acid value typically of 1 to 20, but also higher. Examples of suitable oils—without limitation—are palm oil, palm kernel oil, coconut oil, olive oil, sunflower oil, saflor oil, soy oil, line oil, rape oil, fish oil, lard oil and tallow. The de-acidification of crude vegetable oils preferably is done under vacuum in a counter-current column with stripping steam as shown in FIG. 1:

In a preferred embodiment the crude oil is preheated by economizers (1, 2) and pre-heater (3) and fed to the top of the column (4). While trickling down the column free fatty acids and low boiling impurities like aldehydes, ketones and phenols are stripped from the oil by the stripping steam, which is introduced at the column bottom. The stripped off fatty acids are condensed from the stripping steam in two steps while the de-acidified oil is taken from the column as bottom product.

The column internals are chosen from structured or dumped packing or trays depending on the solids and gum content of the crude oil. For example, in case of coconut or palm kernel oil structured packing is preferred due to low pressure drop. For oils with higher solids/gum content umbrella bubble cap trays are preferred due to lower pressure drop than for other tray types. The height of the packings can be about 3 to about 8 and preferably about 4 to about 6 m. The number of trays may be about 6 to about 18, preferably about 8 to about 14. Partial condensation of the fatty acid vapors is performed by a partial condenser (dephlegmator) or preferably by a direct condensation in a packing (5) with recirculation loop and external plate cooler (6). Vapors leaving the partial condenser are condensed in the second condenser (7). An additional feature of the invention is to install an additional packing (8) above the feed and to run the column with a small reflux of the first distillate in order to reduce the amount of glycerides in the fatty acid distillate. In this case an additional reboiler (9) is required to provide the heat of vaporization for the reflux stream.

The de-acidification column may be operated at a vacuum of about 2 to about 20 and preferably about 5 to about 10 mbar. The crude oil feed may be heated to about 225 to about 280 and preferably about 245 to about 260° C. The stripping steam rate may be adjusted to about 1 to about 5, and preferably about 1 to about 2% of the crude oil feed rate. The resulting acid values of the de-acidified oils are between about 0.02 and about 1, preferably about 0.1 to about 0.5, the resulting water content between about 0.01 and about 0.1, preferably about 0.01 to about 0.03%.

Two different fatty acid qualities with different amounts of glycerides are achieved by adjusting the condenser temperature to about 70 to about 100 and preferably about 80° C. Depending on the process conditions the first distillate of the de-acidification column contains about 1 to about 50% b.w. glycerides. This stream may be subjected to an esterification step described in the next section and subsequently be routed to low pressure transesterification together with the de-acidified oil to convert the remaining glycerides to methylester. Alternatively the fatty acid distillate can be routed to a high pressure transesterification, where esterification of acids and transesterification of glycerides are performed simultaneously. The second distillate normally has to be discarded or can be used for technical applications.

Step b: Esterification of Fatty Acid Distillate

In the second step the esterification of the fatty acid distillate obtained from step (a) takes place. In some embodiments, the esterification of the fatty acid distillate can be done either with an acidic catalyst, for example with an acidic ion exchange catalyst in a fixed bed, or by enzymatic conversion, such as by using CALB lipase in a stirred vessel.

Esterification with Acidic Ion Exchange Catalyst

In this case the esterification is performed with an excess of the lower alcohol, preferably methanol or ethanol in one or two fixed bed reactors (10, 11) filled with an acidic ion exchange catalyst. Molar ratios of alcohol to fatty acids are from about 6:1 to about 9:1. After each reaction step process water is separated from the reaction mixture in evaporators (12) and (13), before feeding to the next process step.

Esterification with CALB Lipase

Alternatively the fatty acids can be esterified by enzymatic treatment with CALB lipase. For this purpose the fatty acids are mixed with alcohol, preferably methanol, water and the enzymes in suitable amounts in a stirred tank, heated up to between about 35 and about 45° C. and reacted for about 20 to about 40 hours, until the acid value has decreased to the required degree. Subsequently, the alcohol and water are separated from the mixture by evaporation as described above.

Depending on the degree of conversion the lower alkyl esters, preferably the alkyl esters, generated by the described two esterification procedures can be routed to the following step (c) or to an additional high pressure transesterification step.

Step c: Low Pressure Transesterification of de-Acidified Crude Oil

The main advantages of the combination of the low pressure transesterification with a de-acidification column are lower catalyst consumption, higher yield due to lower product losses into soap formation and a better phase separation between alkyl ester and glycerol.

The low pressure transesterification can be performed in two stages at temperatures in the range of about 65 to about 90° C. The de-acidified and dried crude oil is mixed with the lower alcohol, preferably methanol, and catalyst, heated to reaction temperature and routed to a first reactor (14). After the first reaction stage the formed glycerol is separated from the reaction mixture by gravity. The reaction mixture is then fed to a second reactor (15) together with additional alcohol and catalyst. Suitable equipments to perform the reactions are e.g. static mixers combined with tube reactors or mixer settlers as shown in FIG. 1. Mixer settlers are advantageous with regard of turndown ratios, since at lower feed rates the mixing efficiency is still good and the conversion even improved, while for tube reactors the mixing efficiency is reduced at lower flow rates. It is also possible to combine the elements, e.g. a mixer-settler with a tube reactor.

The reaction pressure is dependent on reaction temperature due to the vapor pressure of the alcohol and may range from about 1 to about 5 bar. Alcohol:oil ratios are typically in the range of about 0.2 to about 0.35, preferably about 100% in excess compared to stoichiometric consumption for the first reaction step, while for the second reaction step the ratio can be decreased by a factor of about 10.

As far as the transesterification catalyst is concerned alkaline catalysts such as alcoholates of alkaline metals are preferred. For example, a 30% sodium methylate catalyst solution in methanol is typically applied. The ratio catalyst solution:oil can range from about 0.5 to about 1.2% b.w., preferably about 0.7 to about 1.0% b.w. for the first reactor and about 0.05 to about 0.12% b.w., preferably about 0.07 to about 0.1% for the second reactor.

Step d: Methanol Removal from Methyl Ester

Another advantage of the crude oil de-acidification is the low water content of the de-acidified oil. As a result the reaction mixture after low pressure transesterification also has very low water content, allowing the application of a simple evaporation procedure for the removal of alcohol from the alkyl ester and glycerol streams. For the alcohol, preferably methanol recovery from the ester phase double effect evaporation can be applied. The first evaporator (16) is operated at about 1 atm and a temperature of about 90 to about 130° C., the second evaporator (17) at about 80 to about 200 mbar and about 120 to about 155° C. The alcohol content of the alkyl ester after the second evaporator is typically less than 0.5%. The alcohol recovered from the alkyl ester phase has a water content of typically less than 0.2%, so that the alcohol can be recycled to the transesterification without further separation of water. Short chain alkyl esters evaporated together with the methanol are trapped by a condenser and routed back to the feed as recycle stream.

Equipment for Operating the Process According to the Invention

Another aspect of the present invention is directed to a system (i.e. equipment) for conducting a process for obtaining fatty acid C₁-C₄ alkyl esters from unrefined fats and oils comprising
(i) a counter-current column,
(ii) one or more fixed-bed esterification reactors or stirred vessels,
(iii) one or more static mixers, mixer-settlers or tube reactors, and
(iv) one or more evaporators,
   said elements (i) to (iv) are connected and operated as follows:
(a) unrefined fats or oils having an acid value of from 1 to 10 are treated with hot steam in counter-current column (i) to provide a first fraction of free fatty acids and low boiling impurities at the top and a second fraction of de-acidified and de-watered fats or oils at the bottom of the column;
(b) said first fraction is subjected to acidic esterification in a fixed-bed esterification reactor or to enzymatic esterification in a stirred vessel (ii) with a lower C₁-C₄ alcohols in order to provide a first fraction of fatty acid C₁-C₄ alkyl esters;
(c) said second fraction is subjected to a low pressure transesterification conducted in either at least two static mixers, at least two mixer settlers or at least two tube reactors (iii), each of them in line, in order to provide an intermediate fraction of fatty acid C₁-C₄ alkyl esters, C₁-C₄ alcohols and glycerol; and
(d) said intermediate is subjected to a separation process in at least two evaporators (iv), operated in line at different temperatures and different pressures, in order to remove C₁-C₄ alcohols and the glycerol in order to provide a second fraction of C₁-C₄ alkyl esters.

Yet another aspect relates to a system for conducting a process for obtaining fatty acid C₁-C₄ alkyl esters from unrefined fats and oils comprising:
(i) a counter-current column,
(ii) one or more esterification reactors selected from fixed-bed esterification reactor or stirred vessels,
(iii) one or more transesterifcation reactors selected from static mixers, mixer-settlers or tube reactors, and
(iv) one or more evaporators,
wherein said elements (i) to (iv) are connected and operated as follows:
(a) said counter-current column having a steam inlet and an unrefined fats or oils inlet, and the top of said counter-current column is in fluid communication with one or more inlets of one or more esterification reactors and the bottom of said counter-current column is in fluid communication with one or more inlets of one or more transesterification reactors;
(b) said one or more esterification reactors are in fluid communication with said one or more transesterification reactors; and
(c) said one or more transesterification reactors are in fluid communication with said one or more evaporators.

### Examples

The following working examples for obtaining a coconut fatty acid methyl ester from unrefined coconut oil has been conducted in equipment as set out in FIG. 1.

### Example 1

### De-Acidification of Coconut Oil

Raw coconut oil with an acid value of 12 was preheated to 260° C. via a pre-heater and pumped at a flow rate of 200 kg/hr to the top of a column with an internal diameter of 130 mm packed with 4.6 m of a structured packing with a specific surface of 350 m²/m³. The column was run at a top pressure of 10 mbar. Stripping steam was introduced into the bottom of the column at a flow rate of 2.0 kg/hr. The first condenser of the column was adjusted to 80° C., a second condenser to 40° C.

The de-acidified oil had an acid value of 0.14. In the first condenser a fatty acid stream of 10.6 kg/hr was condensed having an acid value of 182.4 and a glyceride content of 16%. In the second condenser 0.6 kg/hr were condensed, having an acid value of 284 and a glyceride content of 0.5%.

### Example 2

### Esterification with Acidic Ion Exchange Catalyst

Coconut fatty acid distillate achieved from the de-acidification step described in example 1 was fed to a static mixer at a flow rate of 2.5 kg/hr, where it was continuously mixed with 1.0 kg/hr methanol. The mixture was preheated to 100° C. via a preheater and subsequently fed to two reaction vessels filled with granular acidic ion exchange catalyst Lewatit K2641, each catalyst bed having a volume of 10 l. In between the two reaction vessels the reaction mixture from the 1^{st} reaction was stripped from water and methanol under vacuum before passing a 2^{nd} static mixer together with 0.3 kg of methanol and entering the 2^{nd} fixed bed reactor. Measured AV after 1^{st} and 2^{nd} reaction stages were 18.6 and 0.98 respectively

### Example 3

### Enzymatic Esterification with CAL-B Lipase

500 g of coconut fatty acid distillate achieved from the de-acidification step described in example 1 were mixed with 100 g methanol, 300 g water and 100 mg Novozym CAL-B lipase in a 1 l heat traced glass vessel and stirred at 30° C. AV of the oil phase was reduced from 182.4 to 30.5 after 17 hours and 12.5 after 45 hours.

### Example 4

### Transesterification

De-acidified coconut oil from example 1 was preheated to 60° C. and fed at a flowrate of 10 kg/hr to a first of two subsequently installed mixer settlers, each of them having a mixing volume of 1 l and a settling zone of 10 l and heated to 60° C. with hot water via a double jacket. 2.5 kg/hr of a mixture with 98.6% methanol, 1.2% sodium methylate and 0.2% water was also preheated to 60° C. and dosed into the first mixing stage. Glycerol generated by the transesterification reaction in the first mixing chamber was separated by gravity from the oil/methyl ester mixture in the first settling zone and continuously discharged from the system. The methyl ester phase from the first settler was fed to the second mixing chamber together with 0.5 kg/hr of a methanol/sodium methylate/water mixture, having the same composition like added to the first mixing stage. Additional glycerol generated by the transesterification reaction in the second mixing chamber was separated by gravity from the methyl ester in the second settling zone and both phases were taken continuously from the second settler.

Conversions measured by GC of the methyl ester phases as (100%—Triglyceride—Diglyceride—Monoglyceride) after the first and second mixer settler stages were 94% and 97% respectively.

### Example 5

### Methanol Recovery

189.6 kg of the methyl ester phase achieved from the transesterification of de-acidified coconut oil as described in example 4 were fed to a 1 m³ stirred vessel equipped with a waterring pump and a heat jacket heated with hot water. The temperature was adjusted to 95° C. and the vacuum to 900 mbar. 11.8 kg of methanol with a water content of 0.17% were recovered by condensation. Residual methanol content in the methyl ester was 0.14%.

1. A process for obtaining fatty acid C₁-C₄ alkyl esters from unrefined fats and oils, the process comprising:
(a) treating unrefined fats or oils having an acid value of from 1 to 20 with hot steam in a counter-current column to provide a first fraction of free fatty acids and low boiling impurities at the top of the column and a second fraction of de-acidified and de-watered fats or oils at the bottom of the column;
(b) esterifying said first with one or more lower C₁-C₄ alcohols in order to provide a third fraction of fatty acid C₁-C₄ alkyl esters;
(c) combining said second and said third fraction and subjecting to a low pressure transesterification in order to provide an intermediate fraction of fatty acid C₁-C₄ alkyl esters, C₁-C₄ alcohols and glycerol; and
(d) subjecting said intermediate fraction to a separation process in order to remove C₁-C₄ alcohols and the glycerol in order to provide a second fraction of C₁-C₄ alkyl esters.

2. The process of claim 1, wherein said fats and oils are selected from the group consisting of palm oil, palm kernel oil, coconut oil, olive oil, sunflower oil, saflor oil, soy oil, line oil, rape oil, fish oil, lard oil and tallow.

3. The process of claim 1, wherein the de-acidification step (a) is conducted at a reduced pressure of 2 to 20 mbar.

4. The process of claim 1, wherein the de-acidification step (a) is conducted at a temperature of 225 to 280° C.

5. The process of claim 1, wherein the esterification step (b) is conducted in the presence of an acidic catalyst or by enzymatic conversion.

6. The process of claim 5, wherein the esterification step (b) is conducted either in the presence of an acidic ion exchange catalyst or a CLAB lipase.

7. The process of claims 1, wherein the transesterification step (c) is conducted in two stages, wherein:
(c1) in a first stage the de-acidified and dried crude oil is mixed with the lower alcohol and catalyst, heated to reaction temperature and routed to a first reactor to obtain an intermediate transesterification product and glycerol, which is separated off, and
(c2) said intermediate transesterification product is then fed to a second reactor together with additional alcohol and catalyst in order to obtain the final transesterification product.

8. The process of claim 1, wherein the transesterification is conducted in a static mixer, a mixer-settler or a tube reactor.

9. The process of claim 1, wherein the transesterification is conducted at a temperature of 65 to 90° C.

10. The process of claim 1, wherein the transesterification is conducted at a pressure of 1 to 5 bar.

11. The process of claim 1, wherein the transesterification involves an alcohol:oil ratio in the range from 0.2 to 0.35.

12. The process of claim 1, wherein the transesterification is conducted in the presence of alkaline catalysts.

13. The process of claim 1, wherein the transesterification is conducted in the presence of alkaline catalysts at a concentration of 0.5 to 1.2% b.w. for the first and 0.05 to 0.12% b.w. for the second reactor, both calculated on the oil.

14. The process of claim 1, wherein the separation step (d) is conducted in two evaporators operating in line at different pressures and temperatures.

15. A system for conducting a process for obtaining fatty acid C₁-C₄ alkyl esters from unrefined fats and oils comprising:
(i) a counter-current column,
(ii) one or more fixed-bed esterification reactor or stirred vessels,
(iii) one or more static mixers, mixer-settlers or tube reactors, and
(iv) one or more evaporators,
wherein said elements (i) to (iv) are connected and operated as follows:
(a) unrefined fats or oils having an acid value of from 1 to 20 are treated with hot steam in counter-current column (i) to provide a first fraction of free fatty acids and low boiling impurities at the top and a second fraction of de-acidified and de-watered fats or oils at the bottom of the column;
(b) said first fraction is subjected to acidic esterification in a fixed-bed esterification reactor or to enzymatic esterification in a stirred vessel (ii) with a lower C₁-C₄ alcohols in order to provide a third fraction of fatty acid C₁-C₄ alkyl esters;
(c) said second and said third fraction are combined and subjected to a low pressure transesterification conducted in either at least two static mixers, at least two mixer settlers or at least two tube reactors (iii), each of them in line, in order to provide an intermediate fraction of fatty acid C₁-C₄ alkyl esters, C₁-C₄ alcohols and glycerol; and
(d) said intermediate is subjected to a separation process in at least two evaporators (iv), operated in line at different temperatures and different pressures, in order to remove C₁-C₄ alcohols and the glycerol in order to provide a second fraction of C₁-C₄ alkyl esters.

16. A system for conducting a process for obtaining fatty acid C₁-C₄ alkyl esters from unrefined fats and oils comprising:
(i) a counter-current column,
(ii) one or more esterification reactors selected from fixed-bed esterification reactor or stirred vessels,
(iii) one or more transesterifcation reactors selected from static mixers, mixer-settlers or tube reactors, and
(iv) one or more evaporators,
wherein said elements (i) to (iv) are connected and operated as follows:
(a) said counter-current column having a steam inlet and an unrefined fats or oils inlet, and the top of said counter-current column is in fluid communication with one or more inlets of one or more esterification reactors and the bottom of said counter-current column is in fluid communication with one or more inlets of one or more transesterification reactors;
(b) said one or more esterification reactors are in fluid communication with said one or more transesterification reactors; and
(c) said one or more transesterification reactors are in fluid communication with said one or more evaporators.
